(19) United States
(12) Patent Application Publication
(10) Pub. No.: US 2009/0024096 A1
(43) Pub. Date: Jan. 22, 2009

(54) IMMOBILIZATION OF DYES AND ANTIMICROBIAL AGENTS ON A MEDICAL DEVICE

(75) Inventors: TON THAT HAI, Round Lake, IL (US); Mark A. Nordhaus, Antioch, IL (US); Vadim V. Krongauz, Bartlett, IL (US); Kent L. Lurvey, Grayslake, IL (US)

Correspondence Address: BAXTER HEALTHCARE CORPORATION, 1 BAXTER PARKWAY, DF2-2E, DEERFIELD, IL, 60015, US

(73) Assignees: BAXTER INTERNATIONAL INC., DEERFIELD, IL (US); BAXTER HEALTHCARE S.A., ZURICH (CH)

(21) Appl. No.: 11/780,876

(22) Filed: Jul. 20, 2007

(51) Int. Cl. A61M 25/00; A61B 17/00; C07D 213/02

(52) U.S. Cl. 604/265; 427/2.28; 546/338; 606/1

(57) ABSTRACT

A method for immobilizing dyes and antimicrobial agents on a porous surface is disclosed and described. The surface may be that of a medical device, such as a catheter, a connector, a drug vial spike, a bag spike, a prosthetic device, an endoscope, and surfaces of an infusion pump. The surfaces may also be one or more of those associated with a dialysis treatment, such as peritoneal dialysis or hemodialysis, where it is important that working surface for the dialysis fluid be sterile. These surfaces include connectors for peritoneal dialysis sets or for hemodialysis sets, bag spikes, dialysis catheters, and so forth. A method for determining whether a surface has been sterilized, and a dye useful in so indicating, is also disclosed.

### RELATED APPLICATIONS

This application is related to another application, entitled MEDICAL FLUID ACCESS DEVICE, Attorney Docket 112713-1206, U.S. patent application Ser. No. ______, which is filed on the same day as the present application, and assigned to the assignee of the present application, the entire contents of which are hereby incorporated by reference. This application is also related to U.S. patent application Ser. No. 11/458,816, filed Jul. 20, 2006, now U.S. Pat. No. ______, entitled Medical Fluid Access Site With Antiseptic Indicator, and U.S. patent application Ser. No. 11/550,643, filed Oct. 18, 2006, of the same title, now U.S. Pat. No. ______, entitled ______, both of which are incorporated by reference in their entirety.

### BACKGROUND

The present disclosure relates generally to methods of immobilizing dyes and antimicrobial agents on a surface, especially a surface of a medical device. In particular, the disclosure relates to methods of treating a polymer surface for better attachment of antimicrobial agents onto the surface, and for the attachment of dyes to the surface. The dyes will change from a first color or appearance to a second color or appearance when they are swabbed with a disinfecting fluid, such as isopropyl alcohol (IPA) or a solution of water and IPA, especially a solution of 70% water/30% IPA.

Polymers are used in many medical devices in the health care industry. These polymers are used to make devices for therapeutic and for diagnostic purposes. For example, connectors for kidney dialysis, such as peritoneal dialysis and hemo-dialysis may be made of polymers. Dialysate fluid containers, access ports, pigtail connectors, spikes, and so forth, are all made from plastics or elastomers. Therapeutic devices such as catheters, drug vial spikes, vascular access devices such as luer access devices, prosthetics, and infusion pumps, are made from polymers. Medical fluid access devices are commonly used in association with medical fluid containers and medical fluid flow systems that are connected to patients or other subjects undergoing diagnostic, therapeutic or other medical procedures. Other diagnostic devices made from polymers, or with significant polymer content meant for contact with tissues of a patient, include stethoscopes, endoscopes, bronchoscopes, and the like. It is important that these devices be sterile when they are to be used in intimate contact with a patient.

Typical of these devices is a vascular access device that allows for the introduction of medication, antibiotics, chemotherapeutic agents, or a myriad of other fluids, to a previously established IV fluid flow system. Alternatively, the access device may be used for withdrawing fluid from the subject for testing or other purposes. The presence of one or more access devices in the IV tubing sets eliminates the need for phlebotomizing the subject repeatedly and allows for immediate administration of medication or other fluids directly into the subject.

Several different types of access devices are well known in the medical field. Although varying in the details of their construction, these devices usually include an access site for introduction or withdrawal of medical fluids through the access device. For instance, such devices can include a housing that defines an access opening for the introduction or withdrawal of medical fluids through the housing, and a resilient valve member or gland that normally closes the access site. Beyond those common features, the design of access sites varies considerably. For example, the valve member may be a solid rubber or latex septum or be made of other elastomeric material that is pierceable by a needle, so that fluid can be injected into or withdrawn from the access device. Alternatively, the valve member may comprise a septum or the like with a preformed but normally closed aperture or slit that is adapted to receive a specially designed blunt cannula therethrough. Other types of access devices are designed for use with connecting apparatus employing standard male luers. Such an access device is commonly referred to as a “luer access device” or “luer-activated device,” or “LAD.” LADS of various forms or designs are illustrated in U.S. Pat. Nos. 6,682,509, 6,669,681, 6,039,302, 5,782,816, 5,730,418, 5,360,413, and 5,242,432, and U.S. Patent Application Publications Nos. 2003/0208165 and 2003/0141477, all of which are hereby incorporated by reference herein.

Before an access device is actually used to introduce or withdraw liquid from a container or a medical fluid flow system or other structure or system, good medical practice dictates that the access site and surrounding area be contacted, usually by wiping or swabbing, with a disinfectant or sterilizing agent such as isopropyl alcohol or the like to reduce the potential for contaminating the fluid flow path and harming the patient. It will be appreciated that a medical fluid flow system, such as an IV administration set, provides a direct avenue into a patient's vascular system. Without proper aseptic techniques by the physician, nurse or other clinician, microbes, bacteria or other pathogens found on the surface of the access device could be introduced into the IV tubing and thus into the patient when fluid is introduced into or withdrawn through the access device. Accordingly, care is required to assure that proper aseptic techniques are used by the healthcare practitioner. This warning applies to many medical devices, especially those in contact with the patient, and especially so for access devices, which like catheters or infusion pumps, access the patient's bodily orifices, especially those of the vascular system.

As described more fully below, the methods for attaching antimicrobial agents and dyes that indicate that proper aseptic techniques have been used, are believed to represent important advances in the safe and efficient administration of health care to patients.

### SUMMARY

One embodiment is a method of coating a surface. The method includes steps of providing a medical device having a porous polymer surface, cleaning the surface of the medical device, providing a plurality of functional groups on the surface, attaching a linking group to the functional group, and attaching a solvatochromic dye or a derivative of the solvatochromic dye to the functional group or to the linking group.

Another embodiment is a method of coating a surface. The method includes steps of cleaning a porous surface of a medical device made from a polymer, treating the surface with a strong acid to provide a plurality of functional groups on the surface, reacting the functional groups with a linking agent to form attachment sites, the linking agent selected from the group consisting of poly(N-succinimidyl acrylate) (PNSA) and polymers with an aldehyde functional group, and attaching a solvatochromic dye, an antimicrobial agent, or an alkyl-amino containing compound selected from the group consisting of peptides, proteins, Factor VIII or other anti-clotting Factor, polysaccharides, polymyxins, hyaluronic acid, heparin, chitosan, condroitin sulfate, and derivatives of each of these, to the attachment sites.

Another embodiment is a polymeric medical device. The polymeric medical device includes a housing of the polymeric medical device, a porous polymer surface atop the medical device, a plurality of attachment sites on the porous upper polymer surface, optionally, a plurality of functional groups attached to the attachment sites, and also includes at least one of: i. a solvatochromic dye or a derivative of the solvatochromic dye; and ii. an antimicrobial compound, attached to the attachment sites or to the functional groups, wherein the porous polymeric surface is configured to reversibly change from a first appearance to a second appearance when the surface is swabbed with a disinfecting solution.

Another embodiment is a medical device. The medical device includes a medical device having a porous surface made from a polymer, a plurality of attachment sites on the surface of the medical device, optionally, a plurality of functional groups attached to the attachment sites, and an antimicrobial compound, attached to the attachment sites or to the functional groups, wherein the antimicrobial compound is configured to be cidal to, or to resist growth of, microorganisms on the surface of the device.

Another embodiment is a medical device. The medical device includes a medical device having a porous surface made from a polymer, a plurality of attachment sites on the surface of the medical device, optionally, a plurality of functional groups attached to the attachment sites, and an alkyl-amino containing compound selected from the group consisting of peptides, proteins, Factor VIII or other anti-clotting Factor, polysaccharides, polymyxins, hyaluronic acid, heparin, chitosan, and derivatives of each of these, to the attachment sites.

Another embodiment is a dye. The dye includes a compound having a structure:

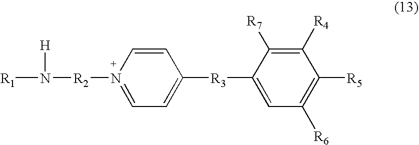

and derivatives thereof, wherein R1 is acryloyl, methacryloyl, or hydrogen, R2 is C4 to C10 alkyl, R3 is ethene, R4 and R6 are bromide, chloride, fluoride, iodide, and mixtures thereof, R5 is one of hydrogen or O⁻, and R7 is the other of hydrogen and O⁻.

Another embodiment is a dye. The dye includes a compound having a structure:

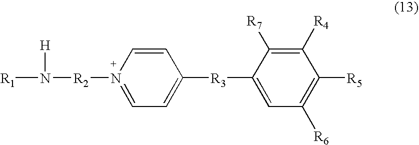

and derivatives thereof, wherein R1 is acryloyl, methacryloyl, hydrogen, halogen, alkoxy, alkyl mercapto, or an aromatic mercaptan, R2 is C4 to C10 alkyl, R3 is ethene, butadiene, or hexatriene, R4 and R6 are bromide, chloride, fluoride, iodide, alkoxy, nitrate, and mixtures thereof, R5 is one of hydrogen or O⁻, and R7 is the other of hydrogen and O⁻.

Another embodiment is a process for making a dye. The process includes steps of reacting a t-butyl-oxycarbonyl (BOC) amino aliphatic alcohol with a sulfonyl halide to yield a BOC-amino-aliphatic-sulfonate, reacting the BOC-amino-aliphatic-sulfonate with 4-picoline to form a pyridinium sulfonate, and reacting the pyridinium sulfonate with a substituted salicylaldehyde compound to form a compound with a merocyanine dye functionality, wherein the merocyanine dye has the general structure of

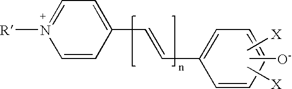

wherein R′=t-butyl-oxycarbonyl, n=1, 2, or 3, X=bromide, chloride, fluoride, iodide, alkoxy, nitrate, and mixtures thereof and are both in meta positions, and wherein the O⁻ is in an ortho or para position.

Another embodiment is a process for making a dye. The process includes steps of forming a BOC-amino-aliphatic-sulfonate from a primary alcohol and a sulfonyl halide, reacting the BOC-amino-aliphatic-sulfonate with 4-picoline to form a pyridium sulfonate, reacting the pyridinium sulfonate with a substituted salicylaldehye to form a phenolate with a monomerocyanine functionality, and dissolving the phenolate in an acid to form a first salt.

Additional features and advantages are described herein, and will be apparent from, the following Detailed Description and the figures.

### BRIEF DESCRIPTION OF THE FIGURES

FIG. 1 is a perspective view of a medical device; and

FIG. 2 is a cross-sectional view of a medical device.

### DETAILED DESCRIPTION

### Synthesis of Solvatochromic Dye Useful as an Antiseptic Indicator

The synthesis of a solvatochromic dye that has been found useful as an antiseptic indicator is herein described. The synthesis was carried out in five distinct steps. A first step reacts 6-t-butyloxycarbonyl-amino-1-hexanol (also known as 6-(BOC-amino)-1-hexanol), compound (1) below, from Sigma Aldrich, St. Louis. Mo., U.S.A., with p-toluenesulfonyl chloride, compound (2) below, to yield 6-(BOC-amino)hexyl-p-toluenesulfonate, compound (3) below.

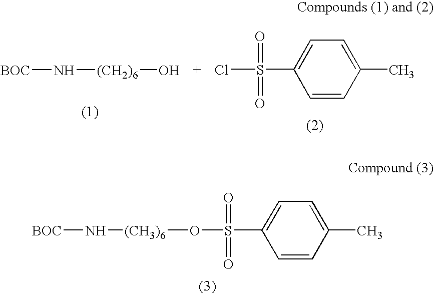

The second step substituted 4-picoline, compound (4) below, for the p-toluene sulfonate portion, resulting in 1-(6-BOC-aminohexyl)-4-methylpyridinium monotosylate, compound (5) below.

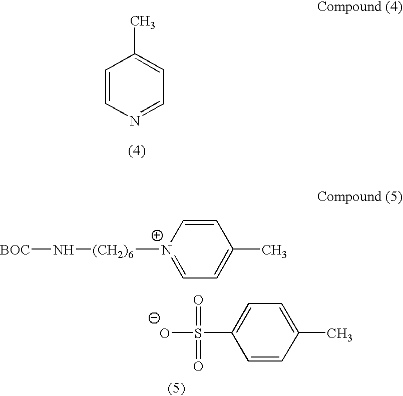

For the third step, 1-(6-BOC-amino)hexyl-4-methylpyridinium was condensed with 3,5-dicholoro-salicylaldehyde, compound 6, in the presence of piperidine, resulting in the formation of 4,6-dichloro-2-[2-(6-BOC-amino)hexyl-4-pyridinio)vinyl]phenolate, compound 7 below.

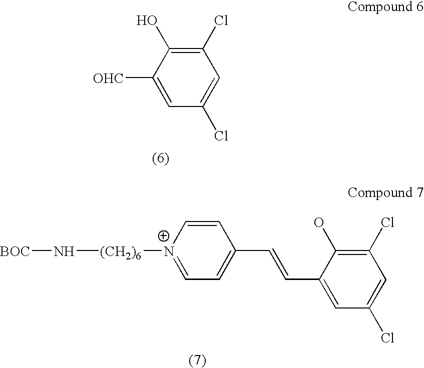

The fourth step then removed the BOC portion by reacting compound 7 with trifluoroacetic acid to yield 4,6-dichloro-2-[2-((6-amino)hexyl-4-pyridinio)-vinyl]phenolate di(trifluoroacetate) salt, compound 8.

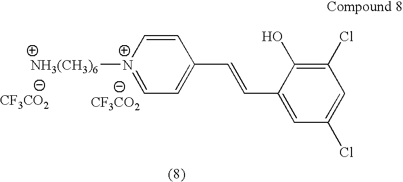

The final step included two parts, the addition of excess acryloyl chloride, compound 9, to form compound 10. This part was followed by hydrolysis of the acryloyl moiety with ammonium hydroxide, which resulted in the dye, compound 11.

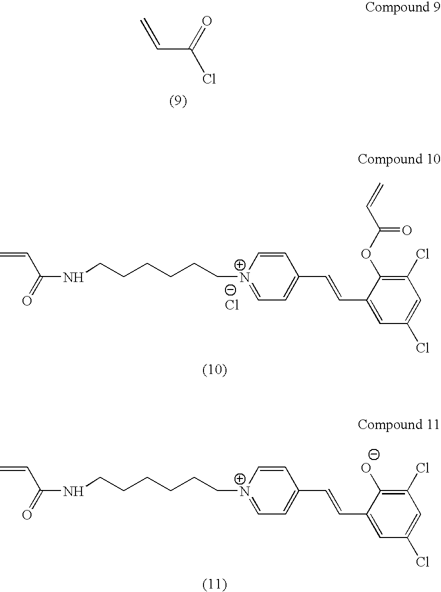

### DETAILED DESCRIPTION OF INDIVIDUAL STEPS

6-t-butyloxycarbonyl-amino-1-hexanol (also known as (BOC-amino)-1-hexanol), compound (1) above, 34.45 grams (hereinafter abbreviated as “g.”), was dissolved in 300 ml chloroform and the solution cooled to about 5° C. in an ice bath while under an argon purge. Triethylamine, 44.2 g. was added and the solution stirred for about 15 minutes. p-Toluene sulfonyl chloride, compound 2, 36.28 g., was added to the solution and the reaction flask was removed from the ice bath and continually stirred for about 4 hours at room temperature. The solution was then concentrated to a clear, slightly yellow oil by rotary evaporation at 30° C. and was azeotroped with 2 sequential extractions with 100 ml chloroform to yield a semi-solid product. The crude product was taken up in 500 ml of a 1:1 mixture of ethyl acetate and hexane, which caused the precipitation of a triethylamine HCl salt, which was removed by filtration. The filter cake was rinsed with 3 sequential rinses of about 75 ml ethyl acetate, which was combined with the filtrate. The filtrate was concentrated to an oil by rotary evaporation at 30° C., yielding about 75 g, and was diluted in 75 ml chloroform. This was purified by flash column chromatography (silica gel) employing a mobile phase solution of hexane: ethyl acetate (5:1 through 1:1). Isolated fractions were then combined and concentrated to yield a white, cloudy oil product, 6-(BOC-amino)hexyl-p-toluenesulfonate, 56.59 g., compound 3 above. The structure was verified with NMR and the mass spectrum (ESI+) peak of 394.2 m/z [M+Na]⁺ is consistent with the sodium salt adduct.

55.63 g. of compound 3 was diluted in 400 ml isopropyl alcohol and 15.4 g. 4-picoline, compound 4 was added while stirring in an argon purge. The reaction solution was heated to reflux and continued for 21 hours reaction time. The solution was then concentrated to a clear, slightly amber-colored oil, 77.64 g. The crude oil product was then diluted in 75 ml chloroform and purified by flash column photography (silica gel) employing a chloroform:methanol (20:1 through 1:1) mobile phase solution. Three sets of isolated fractions were combined and concentrated by rotary evaporation at 30° C.

The first set was a clear yellow oil, 7.57 g., which was relatively impure. The third set was a pure off-white paste, 11.72 g., of 1-(6-BOC-amino)-hexyl)-4-methyl-pyridinium monotosylate, compound (5). The second set was a relatively pure, clear, slightly yellow oil, 45.11 g., which was further purified as follows. It was diluted in 250 ml chloroform, and upon sitting for a few minutes, clear and colorless floating crystals of p-toluenesulfonic acid formed, which were removed by filtration. The filtrate was extracted with three sequential washes of 100 ml distilled water, and the organic layer was then concentrated by rotary evaporation at 30° C. to a clear, slightly yellow oil, yielding 36.88 g. of compound 5. The structure was verified by NMR (nuclear magnetic resonance), and had a mass spectrum (ESI+) with m/z 293.2 [M]⁺, which is consistent with the pyridinium portion of the monotosylate salt that is compound 5.

Compound 5, 46.60 g., was then diluted in 500 ml ethanol and 15.0 ml piperidine was added, followed by 19.16 g. 3,5-dicholorosalicylaldehyde, compound 6. The reaction solution was brought to reflux while stirring under a continuous argon purge. After reacting overnight, the solution was concentrated to a dark purple semi-solid by rotary evaporation at 30° C. This was then dissolved in 200 ml ethanol and distilled water was added drop-wise with rapid stirring. After stirring overnight, the solid, which included both fine and agglomerated particles, was collected. The solid was recrystallized a second time in the same manner. After stirring for 2 hours, a fine orange/red precipitate was collected by filtration. When the filter cake was rinsed with 250 ml distilled water, it immediately turned an olive-green color. The solid was dried overnight at room temperature under a high vacuum. The solid was then recrystallized a third time in the same manner. After stirring for two hours, a fine orange/red precipitate was collected by filtration. The filter cake was rinsed with four sequential washes of 250 ml of a 7:1 mixture of distilled water:ethanol. The solid product was dried at 80° C. at a pressure of about 1 mm Hg for 39 hours, yielding a 36.37 g. of a dark purple solid product, compound 7. The structure was verified by NMR and the mass spectrum (ESI+) m/z of 465.2 [M+H]⁺ was consistent with 4,6-dichloro-2-[2-((6-BOC-amino)hexyl-4-pyridinio)vinyl]phenolate.

10.02 g. of compound 7 was then dissolved in a 1:1 mixture of 100 ml of trifluoroacetic acid and chloroform, and the reaction solution was continuously stirred at room temperature. After 4.5 hours, the reaction was complete and the solution was concentrated to a clear, amber-colored oil. The oil was azeotroped with 3 successive 100 ml aliquots of chloroform, followed by three successive 150 ml aliquots of ethyl acetate, yielding a bright yellow solid product. This product was then taken up in 150 ml ethyl acetate, vigorously shaken, and the fine yellow solid product collected by filtration. The filter cake was rinsed with three successive 25 ml measures of ethyl acetate, and was dried at 50° C. at a pressure of about 1 mm Hg for four hours. The result was 10.73 g. of a bright yellow solid product, 4,6-dichloro-2-[2-((6-amino)hexyl-4-pyridinio)vinyl]phenolate di-(trifluoroacetate) salt, compound 8. The structure was verified by NMR and the mass spectrum (ESI+) m/z of 365.1 [M]⁺, and 183.1 [M+H]²⁺, was consistent with the cationic moiety of compound 8.

This product, 7.07 g., was then dissolved in 200 ml of dimethyl formamide, to which was added a 5 ml solution of 2,6-di-tert-butyl 4-methylphenol, 9.27 mg/ml in 71.1 ml DMF. 10 ml triethylamine was then added, causing the solution to become dark purple. The solution was then cooled to about 5° C., while stirring under an argon purge. Acryloyl chloride, compound 9, in an amount of 3.37 ml in 25 ml chloroform was added dropwise to the solution over a period of about 15 minutes, causing the solution to become clear and light brown in color. After complete addition, the reaction solution was evaluated by thin layer chromatography (TLC) using silica gel F₂₅₄ plates and a chloroform:methanol 2:1 mobile phase. A small amount of acryloyl chloride, about 0.24 ml, was added to the reaction solution and the solution reevaluated later by TLC. The result is believed to be product 10, the chloride salt of 1-acryloyl-4,6-dichloro-2-[2-(1-acrylamidohexyl-4-pyridinio)vinyl]phenolate.

Compound 10 was treated with 15 ml ammonium hydroxide to form the final product. After treatment, 2 L ethyl ether was added to the product with rapid stirring, causing a dark-purple, viscous solid to form. Dark-purple supernatant was decanted from the viscous solid, which was then taken up in 1 L ethyl ether, from which a clear and colorless supernatant was decanted. The solid was mostly dissolved in 100 ml ethanol and 1 L ethyl ether was added to it with rapid stirring. After about 30 minutes, a brownish-purple solid was collected by filtration, and the filter cake was rinsed with ethyl ether. It was then dried under high vacuum for about 2 hours. The product was then purified by flash column chromatography using a 10:1 through 1:1 chloroform:methanol mobile phase solution. Isolated fractions were then combined and concentrated to yield a yellow/orange colored dye. This was then washed with ethyl ether, collected by filtration, and dried under high vacuum overnight, yielding a yellow solid. The solid was dissolved in 150 mL of methanol and 1.5 L of ethyl ether was slowly added with rapid stirring. After 30 minutes, a light green precipitate was collected by filtration and was rinsed with two successive portions of 100 mL ethyl ether. It was dried under high vacuum overnight, yielding 1.085 g. of a light, greenish-yellow solid compound, 11. The structure of compound 11 was verified by NMR and the mass spectrum (ESI+) m/z peak of 419 [M+H]⁺ was consistent with compound 11.

This product, however, did not enjoy solvatochromic activity. It is believed that this was due to stacking and layering of molecules in a tight formation caused by ionic and hydrophobic interactions between adjacent molecules and portions thereof. The product was therefore made basic to restore its dye activity.

The product was then made basic by dissolving 0.8 g. of compound 11 in 20 ml methanol, to which was added 2.00 ml of 1 M NaOH, causing the product to dissolve and form a dark purple color. After stirring for 10 minutes, the solution was concentrated by rotary evaporation at 30° C. to a dark solid. This was redissolved in 20 ml methanol and re-concentrated. It was then azeotroped in three successive aliquots of 25 ml chloroform. The resultant product was then recrystallized by dissolving in 5 ml methanol and adding 100 ml ethyl ether dropwise, while stirring. After about 30 minutes, a fine dark, purple colored solid product precipitated out of solution. This was collected by filtration, washed with ethyl ether, and dried under high vacuum for 11 hours. The result, 0.81 g. of a fine, dark brownish purple solid, was obtained. Other bases may also be used, including at least the hydroxyl compounds of alkali metals, alkaline earths, and ammonium, i.e., potassium hydroxide, calcium hydroxide, ammonium hydroxide, and virtually any other strong hydroxide basic compound.

The result, 4,6-dichloro-2-[2-(1-acrylamidohexyl-4-pyridinio)vinyl]-phenolate, was dissolved in radical polymerizable acrylated resin, discussed elsewhere in this application, in concentrations ranging from 0.1% to 0.5%. The resin was then cured by UV irradiation of 320-350 nm at doses ranging from 0.8 J/cm² to 1.8 J/cm². The result was a solvatochromic film with a bluish-purplish color. When wiped with isopropyl alcohol, the film turned pink, and then returned to a blue color after drying.

While the above description is accurate, it is clear that many modifications may be made to the process and to the end products achieved. For instance, while sodium hydroxide was used to achieve a solvatochromic dye, other bases may also be used for the same purpose, at least the monovalent ones, such as potassium or sodium. Divalent bases, such as calcium or magnesium hydroxide, are also appropriate and work well. It is believed that the more important aspect of making the dye basic is the separation of the molecular layers, rather than the particular cation and base used, e.g., NaOH, NH₄OH, KOH, Mg(OH)₂, Ca(OH)₂, Ba(OH)₂, and so forth, especially bases made with the alkali and alkaline earth metals.

Without being bound to any particular theory, the solvatochromic activity is believed to be due at least in part, to the portion of the molecule between the phenolate ring and the pyridine ring. Accordingly, it has been found that substitution of a hydrogen atom for the acrylamido group does not adversely affect the solvatochromic activity of the dye. The structure of the this molecule, 4,6-dichloro-2-[2-(6-aminohexyl-4-pyridinio)vinyl]phenolate compound 12, is shown below, and is compound 8 discussed above, after neutralization and removal of the trifluoroacetate counterions. In one sense, compound 12 below is compound 11 with a hydrogen substituting for the acryl group.

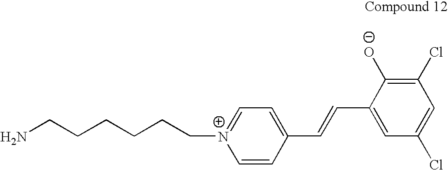

Compound 12 is more easily handled as a salt, which may be the HCl, HBr, HF, phosphate, sulfate, and many others, so long as the species is not carboxylated. In order to make this substance, the compound #8 above is neutralized with a mixture of HCl/dioxane (available from Aldrich) or HCl dissolved in other compatible organic solvent, such as chloroform.

The same compound, with a methacrylamido group, equally activating or electron-withdrawing, is also suitable and may be achieved using methacryloyl chloride in the step for the conversion of compound 8 above. Other substitutes, R1, on the amine group nitrogen atom include at least the halogens, chloride, bromide, fluoride, iodide, and alkyl mercapto. Alkyl mercapto groups, such as ethyl mercapto, and non-bending aromatic bridge groups, such as aromatic mercaptan, are also suitable. It is also possible that at least short chain alkoxy derivatives, such as C3 through C6, especially C3 and C6, are suitable. A hexyl group between the amine group and the pyridine ring worked well. Other short chain aliphatic molecules may also be used in these solvatochromic dyes, such as isohexyl, pentyl, isopentyl, butyl, isobutyl, and decyl and many others, up to C₂₀, i.e., C₄ to C₂₀ aliphatic. It is also believed that aliphatic species are required. Other molecules that will perform well as a solvatochromic dye include substitution of ethene group between the pyridine ring and the benzene ring by conjugated double bonds of butadiene, —C═C—C═C— or hexatriene, —C′C—C═C—C═C—. Other embodiments may include substitutions on the benzene ring, as shown below in structure 13. Either or both of the chlorides at R4, R6, may be replaced by iodide, bromide, or fluoride. The O⁻ group in the 1-position could instead be placed in the 5-position between the chlorides. It is possible that nitrate, —NO₂, alkoxy, such as methoxy, ethoxy, may also yield a solvatochromic dye. Note that a number of substations on the benzene ring are readily available. For example, several salicylaldehyde compounds with halogen atoms in the 3, 5 positions are readily available from manufactures, such as Sigma-Aldrich, St. Louis, Mo., USA. When the salicylaldehyde molecule reacts with its aldehyde functionality to the pyridine ring on structure 5, the 3, 5 positions on the salicylaldehyde molecule become the 4, 6 positions on the phenol/phenolate product formed. Of course, R1 may be amine or acrylamido, R2 is C4 to C20 aliphatic, R3 is ethene, butadiene, or hexatriene, R4 and R6 are as discussed above, and R5 may be one of hydrogen and O⁻ and R7 may be the other of hydrogen and O⁻.

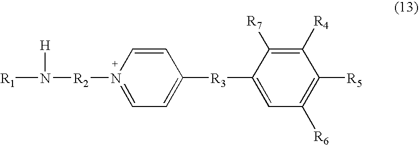

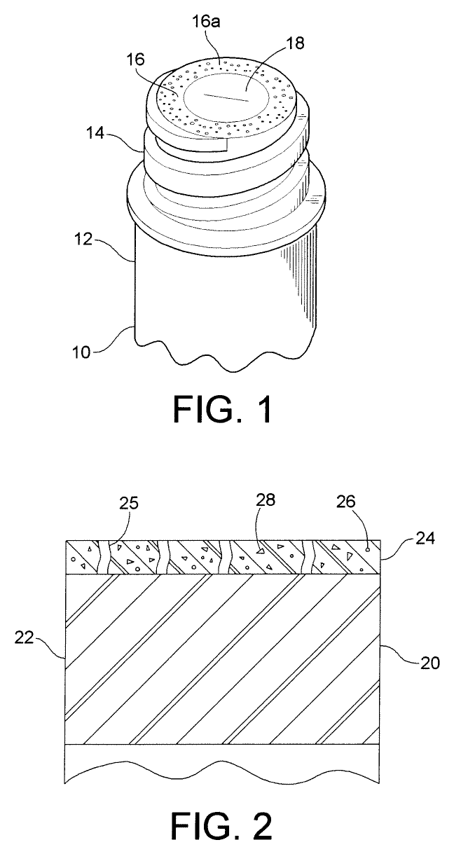

It is possible to incorporate the dye into a coating, preferably a permeable coating, that may be applied to luer access device (LAD) housings. LAD housings are typically made from polycarbonate (PC), but they may also be made from elastomers and other plastics, such as acrylic (such as PMMA), acrylonitrile butadiene styrene (ABS), methyl acrylonitrile butadiene styrene (MABS), polypropylene (PP), cyclic olefin copolymer (COC), polyurethane (PU), polyvinyl chloride (PVC), nylon, and polyester including poly(ethylene terephthalate) (PET). There are many coatings that will firmly adhere to the above mentioned plastics, including epoxies, polyesters, and acrylics. An example of a medical device, a vascular access device, is seen in FIG. 1. Luer access device 10 includes a housing 12, male luer connector threads 14, a rim 16, and a septum 18. Rim 16 is porous and includes a swab-access dye, shown as a dotted surface 16a. Rim 16 and rim surface 16a have been treated so that antimicrobial compounds and dyes will attach to surface 16a.

Other embodiments are described in related application, MEDICAL FLUID ACCESS DEVICE, Attorney Docket 112713-1206, U.S. patent application Ser. No. ______, which is filed on the same day as the present application, and is assigned to the assignee of the present application, the entire contents of which are hereby incorporated by reference. Surface 16a is porous or permeable and the polymer from which the surface is made preferably has an index of refraction from about 1.25 to about 1.6. The permeable surface is typically opaque and may incorporate a small amount of dye. The amount of the dye, such as from about 0.1% to about 1%, is effective in adding a color to the surface, or rendering the surface a translucent with a tint or hint of color.

The surface is porous, so that a disinfecting or antiseptic swabbing solution, such as IPA or a 70% IPA/30% water solution, will permeate the surface. The disinfecting solution may also contain an antimicrobial compound, such as chlorhexidine. If the index of refraction of the swabbing solution, about 1.34, matches or is close to the index of refraction of the polymer from which the porous surface is made, the surface will become transparent, if there is no dye. If a dye is present, the surface will change color as the dye changes state from a first pH to a second, different pH, the pH of the swabbing solution. Solutions or swabbing compounds other than IPA and water may be used, although theses are the most common. For example, ethanol has a refractive index of 1.36. Additions to the swabbing solution, such as chlorhexidine, will also vary the refractive index, thus allowing users to tailor the swabbing solution to insure a visually distinct appearance change, whether from opaque to transparent or from one color to another.

FIG. 2 depicts a medical device 20 with housing 22 and a porous surface layer 24. The pores are shown as narrow channels 25 in the surface layer 24. The porous surface layer may include effective amounts of the dye 26, about 0.1 to about 1.0% by weight, and may also include small amounts of antimicrobial or oligodynamic compounds 28. There are many ways to make compounds porous, e.g., by purchasing membranes with known pore size and density, by applying solvents in the well-known TIPS (thermal inversion phase separation) process, or by inducing surface crazing or cracking into the surface. Polycarbonate membranes with tailored pore sizes may be purchased from Osmonics Corp., Minnetonka, Minn., U.S.A., and polyethylene membranes may be purchased from DSM Solutech, Eindhoven, The Netherlands. Pore sizes may vary from 1 μm down, preferably 0.2 μm down. This small pore size, and smaller, is sufficient to allow permeability to antimicrobial swabbing solutions, but large enough to prevent access by many microorganisms, which tend to be larger than 0.2 μm diameter. Many of these techniques are described in the above-mentioned related patent applications, all of which were previously incorporated by reference.

### Immobilization of Dyes and Microbial Agents on Polymer Surfaces

This section describes the experimental work that was done to prepare such surfaces for direct attachment of the dye molecules. The substances used to prepare the surfaces function by reacting the surfaces and adding functional groups that will bind the dye to the surface. Examples of dyes include Reichardt's dye and the solvatochromic dye described above. As also described above, the dye changes color to alert a medical professional that the surface, such as a luer access device (LAD) surface, has been swabbed and is momentarily clean. This technique is also effective in binding microbial agents to the surface. Examples include chlorhexidine compounds and derivatives, such as chlorhexidine gluconate, and other antimicrobial agents bearing aminoalkyl groups. Examples also include chloroxyphenol, triclosan, triclocarban, and their derivatives, and quaternary ammonium compounds. Many other antimicrobial or oligodynamic substances may also be attached. These compounds are cidal to, or at least to inhibit the growth of, harmful bacteria or other microorganisms on the surfaces to which they are applied, which is beneficial to the patient.

Materials known to have properties of resistance to such microorganisms are described and disclosed in U.S. Pat. No. 4,847,088, U.S. Pat. No. 6,663,877, and U.S. Pat. No. 6,776,824, all of which are hereby incorporated by reference in their entirety as though they were copied directly into this patent. For instance, quaternary ammonium compounds (frequently with organic or silicate side chains) are well-known for such properties, as are boric acid and many carboxylic acids, such as citric acid, benzoic acid, and maleic acid. Pyridinium and phosphonium salts may also be used. Besides organic compounds, certain non-organic materials and compounds are also known for their resistance to germs and organisms. Antimicrobial compounds are used in low concentrations, typically about from about 0.1% to 1% when incorporated into the material itself, e.g., a housing of a luer access device or other vascular access device. Antimicrobial compounds may also be used on many other medical devices, such as catheters, dialysis connects, such as those used in peritoneal dialysis, hemodialysis, or other types of dialysis treatment. They may also be applied to drug vial spikes, prosthetic devices, stethoscopes, endoscopes and similar diagnostic and therapeutic devices, and to infusion pumps and associated hardware and tubing. The use of antimicrobial compounds on these devices, among others, can help to prevent infection and to lessen the effect of infection.

Metals, especially heavy metals, and ionic compounds and salts of these metals, are known to be useful as antimicrobials even in very low concentrations or amounts. These substances are said to have an oligodynamic effect and are considered oligodynamic. The metals include silver, gold, zinc, copper, cerium, gallium, platinum, palladium, rhodium, iridium, ruthenium, osmium, bismuth, and others. Other metals with lower atomic weights also have an inhibiting or cidal effect on microorganisms in very low concentrations. These metals include aluminum, calcium, sodium, lithium, magnesium, potassium, and manganese, among others. For present purposes, all these metals are considered oligodynamic metals, and their compounds and ionic substances are oligodynamic substances. The metals and their compounds and ions, e.g., zinc oxide, silver acetate, silver nitrate, silver chloride, silver iodide, and many others, may inhibit the growth of microorganisms, such as bacteria, viruses, or fungi, or they may have cidal effects on microorganisms, such as bacteria, viruses, or fungi, in higher concentrations. Because many of these compounds and salts are soluble, they may easily be placed into a solution or a coating, which may then be used to coat a vascular access device, such as a luer access device. Silver has long been known to be an effective antimicrobial metal, and is now available in nanoparticle sizes, from companies such as Northern Nanotechnologies, Toronto, Ontario, Canada, and Purest Collids, Inc., Westampton, N.J., U.S.A. Other oligodynamic metals and compounds are also available from these companies.

Other materials, such as sulfanilamide and cephalosporins, are well-known for their resistance properties, including chlorhexidine and its derivatives, ethanol, benzyl alcohol, lysostaphin, benzoic acid analogs, lysine enzyme and metal salt, bacitracin, methicillin, cephalosporin, polymyxin, cefachlor, Cefadroxil, cefamandole nafate, cefazolin, cefime, cefinetazole, cefonioid, cefoperazone, ceforanide, cefotanme, cefotaxime, cefotetan, cefoxitin, cefpodoxime proxetil, ceftaxidime, ceftizomxime, ceftrixzone, cefriaxone moxolactam, cefuroxime, cephalexin, cephalosporin C, cephalosporin C sodium salt, cephalothin, cephalothin sodium salt, cephapirin, cephradine, cefuroximeaxetil, dihydracephaloghin, moxalactam, or loracarbef mafate. Microban, “Additive B,” 5-chloro-2-(2,4 dichloro-phenoxy)phenol is another such material.

### Functional Groups

The following portion discusses a number of processes found to be effective in providing functional groups for the attachment of the above-mentioned solvatochromic dyes and antimicrobial agents. Functional groups may include an activated carboxy group, an activated amine group, or an activated amide group. The desired dye or agent may then be directly attached, or an intermediate group may be used attach the desired substance.

### Nylon Surfaces

In one example, a Whatman nylon-6,6 membrane, pore size 0.2 μm, 47 mm, Whatman Cat. No. 7402-004, was obtained from Whatman Inc., Florham Park, N.J., USA. Other membranes are also available from Whatman, including other nylons or polyamides, polytetrafluoroethylene (PTFE or Teflon®), polyester, polycarbonate, cellulose and polypropylene. The membranes were first washed thoroughly, successively with dichloromethane, acetone, methanol and water. The membranes were then washed several times with water to achieve a neutral pH. They were finally washed in methanol and dried under high vacuum. The membranes were then treated with 3M HCl at 45° C. for four hours to yield specimen NM-1. Without being bound by any particular theory, it is believed that this resulted in the creation of a number of amino groups on the membrane surface. The free amine concentration of the untreated nylon was calculated as 6.37×10⁻⁷ moles/cm², while the free amine concentration after acid treatment was calculated as 13.28×10⁻⁷ moles/cm². The concentration was calculated using the method of Lin et al., described in Biotech Bioeng., vol. 83 (2), 168-173 (2003). Thus, the treatment appeared to double the concentration of free amine on the surface and available for binding.

The NM-1 membrane was then contacted with poly(N-succinimidyl acrylate) (PNSA) dissolved in dimethylformamide (DMF) by placing the membrane in a flask with the dissolved PNSA. It is expected that treatments with other polymers containing aldehyde groups, such as polyacrylaldehyde or polyacrolein, would also be effective. Triethanolamine was then added to the flask, which was rotary shaken while under a continuous argon purge for about 6 hours. The treated nylon membrane was then thoroughly washed with DMF to produce N-succinimidyl carboxylate groups on the surface of the nylon, forming NM-2. The di(trifluoroacetate) salt of 4,6-dichloro-2-[2-(6-amino-hexyl-4-pyridinio)-vinyl]phenolate was dissolved in DMF and was converted by neutralization of the trifluoroacetate counter ions with triethylamine. The previously-treated membrane was added to the reaction flask and was rotary-shaken overnight. The resulting membrane, NM-3, with the salt of 4,6-dichloro-2-[2-(6-amino-hexyl-4-pyridinio)-vinyl]phenolate on its surface, was then thoroughly washed with DMF. The surface of the membrane was a light purple when dry. The same surface turned dark purple when swabbed with isopropyl alcohol, and turned a salmon color when swabbed with a mixture of isopropyl alcohol containing about 30% water.

It is believed that the NM-3 membrane had excess N-succinimidyl carboxylate on its surface. It is also believed that this excess would hydrolyze and protonate the dye at the phenolate position, rendering the dye colorless. A number of NM-3 membranes were treated with different amines to stabilize the carboxy groups and also to discover what colors or other properties would result from the use of different amines. A series of membranes, NM-4 to NM-9 were treated with different amines, resulting in membranes with more stable surfaces but with only slightly different colors. The particular amine was dissolved in methanol, the membrane was added to the reaction flask, and the flask was rotary shaken overnight. The resulting membrane was then washed with acetone and dried under vacuum. Table 1 below summarizes the different used amines and the resulting properties. These results suggest that a number of amino and ammonium compounds may be used to provide attachment sites, including primary amines, ammonium hydroxide, amine (NH₂)-terminated compounds and polymers, morpholine, and an aromatic primary amine.

The membranes had pores on the order of 0.2 μm, resulted in rapid color changes when swabbed, and returned to the dry color within a minute or two. As noted, it is believed that the NM-3 membrane had an excess of carboxylate groups on its surface. Therefore, an antimicrobial agent, chlorhexidine, was applied. Chlorhexidine was dissolved in methanol, the membrane was added to the reaction flask, and the flask was rotary shaken overnight. The membrane was thoroughly washed with acetone and dried under vacuum. It is believed that this membrane, NM-10, now contained both antimicrobial agent and dye. The membrane was tested. Its dry color was a moderate purple, turning to a dark purple in isopropyl alcohol (IPA) and to a moderate orange/red in 70% IPA.

TABLE 1
Amine Treatment of Nylon Membranes

| Nylon | | Amine | | | | | Color, |
|---|---|---|---|---|---|---|---|
| Membrane- | | dose, | reagent | Soln | | Color, | IPA + 30% |
| Number | Amine used | mmol. | soln, ml | pH | Color, dry | IPA | water |
| NM-4 | 2-methoxyethylamine | 15 | 7.50 ml | 11.5 | Very, very | Light | Light |
| | | | DMF | | light pink | brown/ | brown/ |
| | | | | | | pink | pink |
| NM-5 | Hexylamine | 15 | 7.50 ml | 12 | Very, very | Light | Light |
| | | | DMF | | light | brown/ | brown/ |
| | | | | | brown/pink | pink | pink |
| NM-6 | Benzylamine | 15 | 7.50 ml | 11.5 | Very light | Light | Light |
| | | | DMF | | pink | brown/ | brown/ |
| | | | | | | pink | pink |
| NM-7 | Morpholine* | 15 | 7.50 ml | 10 | Moderate | Dark | Salmon |
| | | | DMF | | purple | purple | |
| NM-8 | Ammonium hyroxide | excess | 20 ml | ND** | Moderate | Dark | Salmon |
| | | | NH₄OH | | purple | purple | |
| NM-9 | 3-aminopropyl- | 3.51 | 10 ml | 10 | Light | Moderate | Moderate |
| | terminated poly- | | toluene | | purple | purple | salmon |
| | dimethylsiloxane | | | | | | |

*NM-7 had an additional 0.1 ml triethylamine added, with a final pH of 11- to 11.5.
**The pH of the NM-8 solution was not determined.

### Polycarbonate Surfaces

A second series of plastic surfaces was also tested. DE1-1D Makrofol® polycarbonate films, 0.005 inch thick, clear-gloss/gloss, were obtained from Bayer Polymers Division, Bayer Films Americas, Berlin, Conn., USA. The films were cut into 1 cm squares and were treated with 4 ml of a solution of 0.25 M chlorosulfonic acid in ethyl ether. The square and the solution were placed in a screw-cap vial and cooled to about 5° C. and rotary shaken for 1 hour. The resulting chlorosulfonated film was thoroughly washed with ethyl ether to yield membrane PC-1. It is believed that the amino end groups on the 4,6-dichloro-2-[2-(6-amino-hexyl-4-pyridinio)-vinyl]phenolate dye would react with the chlorosulfonyl groups which had been attached to the polycarbonate surface. A solution of the dye was prepared by dissolving 10 mmol in ethanol and treating with 0.22 mmol triethylamine. The resulting dye solution had a pH of 9.7. The PC-1 film was then added to a rotary flask containing the dye and was rotary shaken overnight and then washed thoroughly with methanol to yield film PC-2. The dry film had a moderately pinkish/purple color. When wetted with 70% IPA, it turned to a peach color.

Other films treated in the same manner, but with a four-hour chlorosulfonic acid treatment, had no color change activity. It is believed that the chlorosulfonyl moiety is a temporary transition product that converts to a more stable entity over time, and thus is not available for attachment of the dye. Other experiments included varying the time for dye attachment from 1 day to 5 days. The films treated for longer periods of time also had more intensely-colored surfaces. Due to the solubility of PC in other solvent, only ethyl ether was used for this experiment. The color change in the polycarbonate film, with very low porosity, was much slower than the color change in membranes, which have a high and regulated porosity. Treatment of polycarbonate surfaces with methacrylic acid or acrylic acid is expected to add carboxyl function groups to the surface.

### Polyester Surfaces

Polyester surfaces were also obtained and tested, e.g., Millipore polyethyleneterephthalate (PET) membranes were obtained, Cat. No. T6PN1426, from Millipore Corp., Billerica, Mass., USA. These membranes were 47 mm in diameter, 0.013 mm thick, with pores having a nominal diameter of 1.0 μm. The membranes were cut into 3 cm×3 cm squares and added to a solution of water and acetone in a screw-cap bottle. 7.5 mmol of methacrylic acid, followed by 0.090 mmol of benzoyl peroxide in 2 ml acetone, were added to the solution. The bottle was rotary shaken at 85 C for 4 hours. The resulting membrane was thoroughly washed several times with hot water, followed by acetone, and then dried under vacuum to yield membrane PET-1. Without being bound to any particular theory, it is believed that this treatment results in substitution of a benzene ring hydrogen in the terephthalate moiety by the acrylic functionality. The membranes were tested, and treatment by acrylic acid resulted in weight gains of 50-53 percent. It is also believed that the subsequent treatment with benzoyl peroxide results in attachment of carboxyl groups to the polyester or PET surface. At least some of the attachments may be of a polymeric rather than monomeric nature, i.e., the attachments may be at least short chains with multiple carboxyl terminations. The terminal amine groups of the 4,6-dichloro-2-[2-(6-amino-hexyl-4-pyridinio)vinyl]phenolate dye, or of an antimicrobial agent, can then attach to the carboxyl groups, with the elimination of water.

A solution of the dye was prepared as follows for the PET membranes. 0.25 mmol of the di(trifluoroacetate) salt was dissolved in 10 ml of DMF, to which was added 0.51 mmol of triethylamine. 0.30 mmol of EEDQ (2-ethoxy-1-ethoxycarbonyl-1,2 dihydroquinoline) coupling agent was added. The PET-1 membrane was added to this reaction solution and was rotary shaken overnight. The resulting membrane was thoroughly washed with methanol. This membrane had a light orange/red color. It is believed that the residual carboxyl groups may protonate the phenolate moiety of the dye, rendering it colorless. Therefore, the membrane was surface-treated with a 5% sodium bicarbonate solution to convert any remaining carboxy groups to the sodium salt. The membrane was then washed with water, followed by methanol, and dried under vacuum to yield the PET-2 membrane. The dry film was orange/red. When wetted with 70% IPA, the membrane became a light salmon color, and changed to a salmon color when tested with IPA alone. In further experiments, it was found that increasing the treatment time of the membrane by the dye solution caused a more intense coloration of the membrane.

The results of these tests demonstrate that several substrates are suitable for the attachment of solvatochromic dyes, or may be treated so that the dyes easily attach. In addition to the particular materials tested, urethane membranes and foams may be used, perhaps without any treatment because of the NHCOO functional groups inherent in urethanes. These results demonstrate that discrete, small rings or membranes, such as those cut from a sheet, may be used. Other polymeric surfaces useful in embodiments include thin films, cast films, molded or shaped parts, or even thin coatings intended for placement on another object, for example, a vascular access device, such as a luer access device.

As discussed above, acrylic membranes or coatings may be used, at least for Reichardt's dye without treatment. The presence of polyester-like RCOO groups in acrylic polymers renders them suitable from the start for attachment of amine-containing dyes or antimicrobials, as well as other dyes. Urethane membranes or foams may be used as is, or they may be treated to make them even more suitable for dye or antimicrobial attachment. Polyimides may suitable if they are flame- or plasma treated, or if foamed polyimides are used. Melamines, maleic anhydride derivatives, blends and co-polymers may also be useful, as may blends, co-polymers and composites of any of these materials. Silicones are less amenable to treatment, however, foamed silicones may be used. For example, treating silicone with 5-10 M NaOH for several hours forms Si—OH (silanol) groups, which can then be used to form carboxy or other functional group attachment sites.

### Solvatochromic Dyes

The dyes described above, Reichardt's dye, 4,6-dichloro-2-[2-(6-acrylamido-hexyl-4-pyridinio)vinyl]phenolate, and 4,6-dichloro-2-[2-(6-amino-hexyl-4-pyridinio)vinyl]phenolate, are only a few of many examples of useful solvatochromic dyes that may be used in these applications. There are many other solvatochromic dyes that could be used. As noted above, the principal requirements are the ability to reversibly change color when swabbed, e.g., with IPA. Without being bound to any particular theory, it is believed that the conjugation between the pyridine ring and the benzene ring, with the intermediary double bond, whether one, two, or three, that accounts for the solvatochromic activity in the new structures. Since these structural features are present in merocyanine dyes, it is believed that a number of these dyes would also be effective as indicators for swabbing, whether incorporated into a coating, as the acrylics described above, or used as part of a surface treatment. Of course, merocyanine dyes typically have a phenoxide ring, rather than a substituted benzene ring. The phenoxide ring functions as the aromatic donor and the pyridine or pyridinium ring functions as the acceptor. Of course, in the new structures, the benzene ring is the donor and the pyridine ring is the acceptor. Thus, it is believed that merocyanine dyes, structure 14 below, with conjugated pyridinium-phenoxide rings (having resonance with a pyridine-benzene structure)

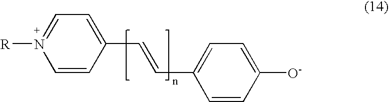

are also suitable. Examples include 1-methyl-4-(4′-hydroxybutyl)pyridinium betaine and Brooker's merocyanine dye, 4′-hydroxy-1-methylstilbaxolium betaine.

Other solvatochromic dyes may also be used, such as an abundance of previously-known dyes, and for which the small change from their normal environment to a slightly acidic environment, such as the 6-7 pH range of IPA, will produce a color change. The table below lists a number of these dyes and their colors before and after. Note that the “before” environment of the coating or LAD housing material may be altered, such as by making it basic, by simple adjustments during the formation of the coating, the method of treating the surface, or the species used for attaching the dye. A few examples of solvatochromic dyes are presented in Table 2 below.

TABLE 2
Solvatochromic Dyes

| | First state | | Second | |
|---|---|---|---|---|
| Dye | pH | Color | state, pH | Color |
| Bromocresol purple | 6.8 | blue | 5.2 | yellow |
| Bromothymol blue | 7.6 | blue | 6.0 | yellow |
| Phenol red | 6.8 | yellow | 8.2 | red |
| Cresol red | 7.2 | red | 8.8 | Red/purple |
| Methyl red | 4.2 | pink | 6.2 | yellow |
| Reichardt's Dye | Unk | green | 6-7 | dark blue |
| Morin hydrate | 6.8 | red | 8.0 | yellow |
| Disperse orange 25 | 5.0 | yellow | 6.8 | pink |
| Nile red | Unk | Blue/purple | 6-7 | bright pink |

These and many other solvatochromic and merocyanine dyes many be used in applications according to this application. Other solvatochromic dyes include, but are not limited to, pyrene, 4-dicyanmethylene-2-methyl-6-(p-dimethylaminostyryl)-4H-pyran; 6-propionyl-2-(dimethylamino)naphthalene; 9-(diethylamino)-5H-benzo[a]phenoxazin-5-one; phenol blue; stilbazolium dyes; coumarin dyes; ketocyanine dyes, Reichardt's dyes; thymol blue, congo red, methyl orange, bromocresol green, methyl red, bromocresol purple, bromothymol blue, cresol red, phenolphthalein, seminaphthofluorescein (SNAFL) dyes, seminaphtharhodafluor (SNARF) dyes, 8-hydroxypyrene-1,3,6-trisulfonic acid, fluorescein and its derivatives, oregon green, and a variety of dyes mostly used as laser dyes including rhodamine dyes, styryl dyes, cyanine dyes, and a large variety of other dyes. Still other solvatochromic dyes may include indigo, 4-dicyanmethylene-2-methyl-6-(p-dimethylaminostyryl)-4H-pyran (DCM); 6-propionyl-2-(dimethylamino)naphthalene (PRODAN); 9-(diethylamino)-5H-benzo[a]phenox-azin-5-one (Nile Red); 4-(dicyanovinyl)julolidine (DCVJ); phenol blue; stilbazolium dyes; coumarin dyes; ketocyanine dyes; N,N-dimethyl-4-nitroaniline (NDMNA) and N-methyl-2-nitroaniline (NM2NA); Nile blue; 1-anilinonaphthalene-8-sulfonic acid (1,8-ANS), and dapoxylbutylsulfonamide (DBS) and other dapoxyl analogs. Other suitable dyes that may be used in the present disclosure include, but are not limited to, 4-[2-N-substituted-(1,4-hydropyridin-4-ylidine)ethylidene]cyclohexa-2,5-di-en-1-one, red pyrazolone dyes, azomethine dyes, indoaniline dyes, and mixtures thereof.

Other merocyanine dyes include, but are not limited to, Merocyanine dyes (e.g., mono-, di-, and tri-merocyanines) are one example of a type of solvatochromic dye that may be employed in the present disclosure. Merocyanine dyes, such as merocyanine 540, fall within the donor—simple acceptor chromogen classification of Griffiths as discussed in “Colour and Constitution of Organic Molecules” Academic Press, London (1976). More specifically, merocyanine dyes have a basic nucleus and acidic nucleus separated by a conjugated chain having an even number of methine carbons. Such dyes possess a carbonyl group that acts as an electron acceptor moiety. The electron acceptor is conjugated to an electron donating group, such as a hydroxyl or amino group. The merocyanine dyes may be cyclic or acyclic (e.g., vinylalogous amides of cyclic merocyanine dyes). For example, cyclic merocyanine dyes generally have the following structure 15, in association with structure 14 above:

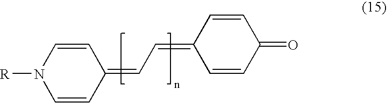

wherein, n is an integer, including 0. As indicated above by the general structures 14 and 15, merocyanine dyes typically have a charge separated (i.e., “zwitterionic”) resonance form. Zwitterionic dyes are those that contain both positive and negative charges and are net neutral, but highly charged. Without intending to be limited by theory, it is believed that the zwitterionic form contributes significantly to the ground state of the dye. The color produced by such dyes thus depends on the molecular polarity difference between the ground and excited state of the dye. One particular example of a merocyanine dye that has a ground state more polar than the excited state is set forth above as structures 14 and 15.

The charge-separated left hand canonical 14 is a major contributor to the ground state, whereas the right hand canonical 15 is a major contributor to the first excited state. Still other examples of suitable merocyanine dyes are set forth below in the following structures 19-29, wherein, “R” is a group, such as methyl, alkyl, aryl, phenyl, etc. See Structures 19-29 below.

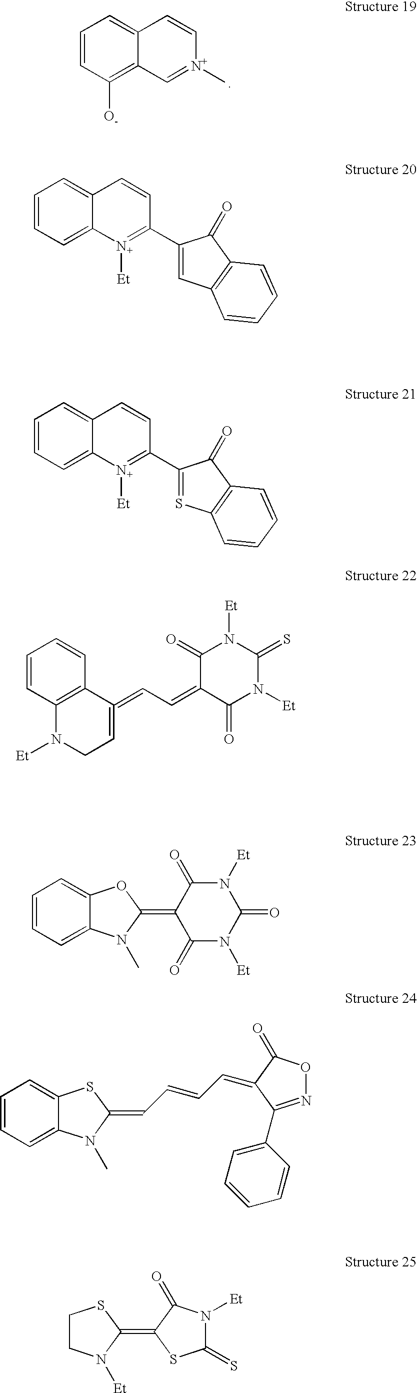

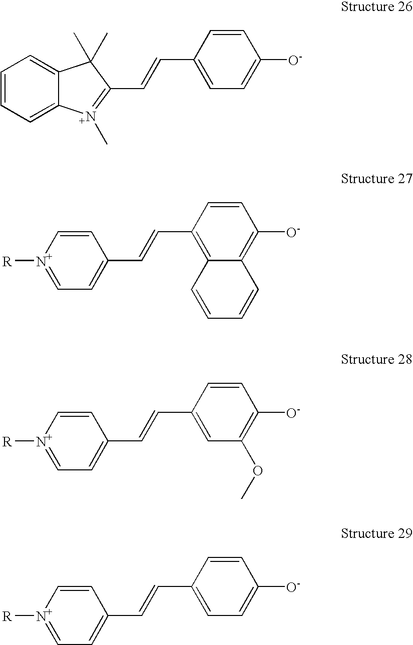

In addition to dyes and antimicrobial compounds, the preparations discussed herein may be used to attach to desired surfaces other compounds or substances containing amino alkyl groups. Examples of these types of compounds include poly(ethylene glycol) (PEG)-containing amino alkyl groups, peptides including antimicrobial peptides, proteins, Factor VIII, polysaccharides such as heparin, chitosan, hyaluronic acid derivatives containing amino alkyl groups, and condroitin sulfate derivates containing amino alkyl groups. One example of a protein is albumin, and an example of a peptide is polymyxin. The one thing these compounds have in common is an amino alkyl group, such as the amino alkyl group discussed above in the new dye, 4,6-dichloro-2-[2-(6-aminohexyl-4-pyridinio)vinyl]phenolate.

Per the discussion above for surface preparation, the same preparation used to attach dyes and antimicrobial compounds containing alkyl amino groups will be suitable for these additional compounds. The amino alkyl groups will bind to the N-succinimidyl carboxylate groups. One technique for treating these groups is to clean the surface, followed by treatment with acid at elevated temperature, and then contacting the surface with poly(N-succinimidyl)acrylate (PNSA). It is believed that this induces carboxylate groups on the nylon surface, suitable for binding to aminoalkyl groups. Other methods are also described. For polycarbonate surfaces, treating with chlorosulfonic acid followed by washing is believed to induce chlorosulfonyl groups. These are suitable for binding by aminoalkyl groups. The treatment above of the PET surfaces is believed to result in attachment of carboxyl groups to the surface, making the also suitable for attachment of aminoalkyl groups.

Thus, polymeric surfaces as described above may also be used for attachment of peptides, proteins, Factor VIII or other anti-clotting Factors, polysaccharides, polymyxins, hyaluronic acid, heparin, chitosan, condroitin sulfate, and derivatives of each of these.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A method of coating a surface, the method comprising:
providing a medical device having a porous polymer surface;
cleaning the surface of the medical device;
providing a plurality of functional groups on the surface;
attaching a linking group to the functional group; and
attaching a solvatochromic dye or a derivative of the solvatochromic dye to the functional group or to the linking group.

2. The method of claim 1, further comprising attaching an effective amount of an antimicrobial agent to the functional group or to the linking group.

3. The method of claim 1, wherein the functional groups on the surface are provided by reacting the surface with an acid, washing, and drying.

4. The method of claim 1, wherein the linking group is provided by poly(N-succinimidyl acrylate) (PNSA) or a polymer with an aldehyde functional group.

5. The method of claim 1, further comprising masking the polymer surface and directing at least the solvatochromic dye or the derivative of the dye, to a desired location on the porous surface.

6. The method of claim 1, further comprising swabbing the porous polymer surface with a disinfecting solution, whereupon a color or an appearance of the surface changes reversibly.

7. The method of claim 6, further comprising allowing the disinfecting solution to evaporate, whereupon the color or the appearance of the porous polymer surface changes back to the color or the appearance that existed before swabbing.

8. The method of claim 7, wherein the porous polymer surface is a membrane or a coating.

9. The method of claim 1, wherein the porous polymer surface is made from a polymer having an index of refraction from about 1.25 to about 1.6.

10. The method of claim 1, further comprising attaching an effective amount of an alkyl-amino containing compound selected from the group consisting of heparin, proteins, chitosan, Factor VIII or other anti-clotting Factor, polysaccharides, peptides, polymyxins, hyaluronic acid, condroitin sulfate, and derivatives of each of these.

11. A method of coating a surface, comprising:
cleaning a porous surface of a medical device made from a polymer;
treating the surface with a strong acid to provide a plurality of functional groups on the surface;
reacting the functional groups with a linking agent to form attachment sites, the linking agent selected from the group consisting of poly(N-succinimidyl acrylate) (PNSA) and polymers with an aldehyde functional group; and
attaching a solvatochromic dye, an antimicrobial agent, or an alkyl-amino containing compound selected from the group consisting of peptides, proteins, Factor VIII or other anti-clotting Factor, polysaccharides, polymyxins, hyaluronic acid, heparin, chitosan, and derivatives of each of these, to the attachment sites.

12. The method of claim 11, wherein the polymer has an index of refraction from about 1.25 to about 1.6.

13. The method of claim 11, further comprising treating the surface to induce amine functional groups.

14. The method of claim 11, wherein the solvatochromatic dye is selected from the group consisting of 4,6-dichloro-2-[2-(6-aminohexyl-4-pyridinio)-vinyl]phenolate and derivatives, Reichardt's dye, its salts and derivatives, and merocyanine dyes and their derivatives.

15. The method of claim 11, further comprising stabilizing the surface by converting unreacted carboxy attachment sites to a salt.

16. The method of claim 11, wherein the surface comprises a membrane or a coating for attachment to the medical device.

17. The method of claim 11, wherein treating a nylon surface with a strong acid results in amino attachment sites, treating a polycarbonate surface with chlorosulfonic acid results in sulfonyl chloride attachment sites, and treating a polyester surface or polycarbonate surface with an acrylic or methacrylic acid results in carboxy attachment sites.

18. A polymeric medical device, comprising:
a housing of the polymeric medical device;
a porous polymer surface atop the medical device;
a plurality of attachment sites on the porous upper polymer surface;
optionally, a plurality of functional groups attached to the attachment sites; and
at least one of: i. a solvatochromic dye or a derivative of the solvatochromic dye; and ii. an antimicrobial compound, attached to the attachment sites or to the functional groups, wherein the porous polymeric surface is configured to reversibly change from a first appearance to a second appearance when the surface is swabbed with a disinfecting solution.

19. The medical device according to claim 18, wherein the polymer surface is made from a polymer having an index of refraction from about 1.25 to about 1.6.

20. The medical device according to claim 18, wherein the porous upper polymer surface is a discrete membrane cut from a sheet, a foamed article, a thin film, a casting, a molding, or a coating.

21. The medical device according to claim 18, wherein the antimicrobial compound comprises an effective amount of a compound selected from the group consisting of chlorhexidine its salts and derivates, an antimicrobial agent bearing an aminoalkyl group, chloroxyphenol, triclosan and triclocarban and derivatives, and a quaternary ammonium compound.

22. The medical device surface according to claim 18, wherein the housing further comprises an effective amount of an oligodynamic compound or an antimicrobial compound.

23. A medical device, comprising:
a medical device having a porous surface made from a polymer;
a plurality of attachment sites on the surface of the medical device;
optionally, a plurality of functional groups attached to the attachment sites; and
an antimicrobial compound, attached to the attachment sites or to the functional groups, wherein the antimicrobial compound is configured to be cidal to, or to resist growth of, microorganisms on the surface of the device.

24. The medical device according to claim 23, wherein the medical device is selected from the group consisting of catheters, drug vial spikes, connectors, vascular access devices, luer access devices, access ports, medication ports, pigtail connectors, prosthetics, endoscopes, bronchoscopes, stethoscopes, and infusion pumps.

25. The medical device according to claim 23, wherein the porous surface is made from a polymer having an index of refraction from about 1.25 to about 1.6 and is configured to change a color or an appearance when the surface is swabbed with a disinfecting solution.

26. The medical device according to claim 23, wherein the porous surface further comprises a solvatochromic dye or a salt or a derivative thereof in an amount from about 0.1% to about 0.5% of the weight of the porous surface.

27. The medical device of claim 23, wherein the polymer is selected from the group consisting of elastomers, acrylic, COC, nylon, methacrylic, elastomer, polycarbonate, polyurethane, polyester, and vinyl-ester.

28. The medical device according to claim 23, wherein the attachment sites comprise one of carboxy groups, amine groups, and amide groups.

29. The medical device according to claim 23, wherein the surface comprises a discrete membrane cut from a sheet, a foamed article, a thin film, a casting, a molding, or a coating.

30. The medical device according to claim 23, wherein the antimicrobial compound comprises an effective amount of compound selected from the group consisting of chlorhexidine, its salts and derivates, an antimicrobial agent bearing an aminoalkyl group, chloroxyphenol, triclosan and triclocarban and derivatives, and a quaternary ammonium compound.

31. The medical device according to claim 23, wherein the surface further comprises an effective amount of an oligodynamic or an antimicrobial material.

32. A medical device, comprising:
a medical device having a porous surface made from a polymer;
a plurality of attachment sites on the surface of the medical device;
optionally, a plurality of functional groups attached to the attachment sites; and
an alkyl-amino containing compound selected from the group consisting of peptides, proteins, Factor VIII or other anti-clotting Factor, polysaccharides, polymyxins, hyaluronic acid, heparin, condroitin sulfate, chitosan, and derivatives of each of these, to the attachment sites.

33. The medical device according to claim 32, further comprising an antimicrobial compound, attached to the attachment sites or to the functional groups, wherein the antimicrobial compound is configured to be cidal to, or to resist growth of, microorganisms on the surface of the device.

34. The medical device according to claim 32, further comprising a solvatochromic dye or a derivative of the solvatochromic dye attached to the attachment sites or to the functional groups.

35. The medical device according to claim 32, wherein the medical device is selected from the group consisting of catheters, drug vial spikes, connectors, vascular access devices, luer access devices, access ports, medication ports, pigtail connectors, prosthetics, endoscopes, bronchoscopes, stethoscopes, and infusion pumps.

36. A dye, comprising:
a compound having a structure 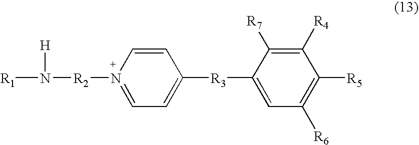
and derivatives thereof, wherein R1 is acryloyl, methacryloyl, or hydrogen, R2 is C4 to C10 alkyl, R3 is ethene, R4 and R6 are bromide, chloride, fluoride, iodide, and mixtures thereof, R5 is one of hydrogen or O⁻, and R7 is the other of hydrogen and O⁻.

37. The dye according to claim 36, wherein if R1 is acryloyl, the derivatives comprise ammonium hydroxide, alkali and alkaline earth salts, and mixtures thereof, and if R1 is hydrogen, the derivatives comprise a hydrobromide, hydrochloride, hydrofluoride, phosphate, sulfate, and mixtures thereof.

38. The dye according to claim 36, wherein R1 is hydrogen, R2 is n-hexyl, R4 and R6 are chloride, R5 is hydrogen, and R7 is O⁻.

39. The dye according to claim 36, further comprising a medical access device in which the dye is present in a porous polymer at about 0.1 to about 0.5% as a swabbing indicator.

40. A dye, comprising:
a compound having a structure 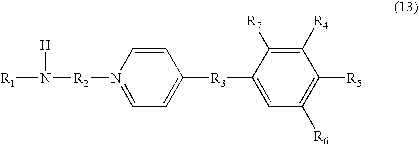
and derivatives thereof, wherein R1 is acryloyl, methacryloyl, hydrogen, halogen, alkoxy, alkyl mercapto, or an aromatic mercaptan, R2 is C4 to C10 alkyl, R3 is ethene, butadiene, or hexatriene, R4 and R6 are bromide, chloride, fluoride, iodide, alkoxy, nitrate, and mixtures thereof, R5 is one of hydrogen or O⁻, and R7 is the other of hydrogen and O⁻.

41. The dye according to claim 40, further comprising a medical access device in which the dye is present on a porous surface of the device or in a porous coating in about 0.1% to about 0.5% as a swabbing indicator.

42. The dye according to claim 40, further comprising a medical access device in which the dye is present on the device, in a porous membrane attached to the device, or as part of a porous surface of the device.

43. A process for making a dye, comprising:
reacting a t-butyl-oxycarbonyl (BOC) amino aliphatic alcohol with a sulfonyl halide to yield a BOC-amino-aliphatic-sulfonate;
reacting the BOC-amino-aliphatic-sulfonate with 4-picoline to form a pyridinium sulfonate; and
reacting the pyridinium sulfonate with a substituted salicylaldehyde compound to form a compound with a merocyanine dye functionality, wherein the merocyanine dye has the general structure of 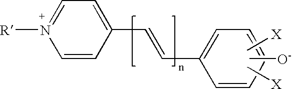
wherein R′=t-butyl-oxycarbonyl, n=1, 2, or 3, X=bromide, chloride, fluoride, iodide, alkoxy, nitrate, and mixtures thereof and are both in meta positions, and wherein the O⁻ is in an ortho or para position.

44. The process of claim 43, further comprising dissolving the merocyanine dye in acid to form a salt.

45. The process of claim 43, wherein the BOC amino aliphatic alcohol is 6-(BOC-amino)-1-hexanol.

46. The process of claim 43, wherein the BOC amino aliphatic alcohol is a saturated aliphatic alcohol having from 4 to 20 carbon atoms, and having an alcohol function group on one end and a BOC-amino functional group on an opposite end.

47. The process of claim 43, wherein the sulfonyl halide is selected from the group consisting of p-toluenesulfonyl chloride and p-toluenesulfonyl bromide.

48. The process of claim 43, wherein the pyridinium sulfonate comprises 1-(6-BOC-amino)hexyl-4-methyl-pyridinium monotosylate.

49. The process of claim 43, wherein the salicylaldehyde comprises two halogen atoms at 3, 5 positions from a position of an aldehyde functional group on the salicylaldehyde.

50. The process of claim 43, further comprising reacting the compound formed in claim 43 with acrylol chloride or methacryloyl chloride to form a structure, wherein R″ is hydrogen or methyl:

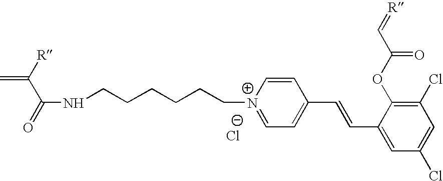

51. The process of claim 50, further comprising hydrolyzing the compound formed in claim 50 with a strong base to form a salt.

52. The process of claim 51, further comprising mixing the compound with a plastic formulation.

53. The process of claim 51, further comprising mixing the compound with a plastic formulation in an amount from about 0.1% to about 0.5% by weight.

54. A process for making a dye, the process comprising:
forming a BOC-amino-aliphatic-sulfonate from a primary alcohol and a sulfonyl halide;
reacting the BOC-amino-aliphatic-sulfonate with 4-picoline to form a pyridium sulfonate;
reacting the pyridinium sulfonate with a substituted salicylaldehye to form a phenolate with a monomerocyanine functionality; and
dissolving the phenolate in an acid to form a first salt.

55. The process according to claim 54, further comprising dissolving the salt, reacting the mixture with acryloyl chloride or methacryloyl chloride, and hydrolyzing the solution in a strong base to form a second salt.

56. The process according to claim 54, further comprising mixing the salt with a plastic formulation in an amount from about 0.1% to about 0.5% by weight.
